(19) United States
(12) Patent Application Publication
(10) Pub. No.: US 2004/0009516 A1
(43) Pub. Date: Jan. 15, 2004

(54) Arrayed SPR prism

(76) Inventors: Bryce P. Nelson, Madison, WI (US); Todd C. Strother, Grove, WI (US)

Correspondence Address: MEDLEN & CARROLL, LLP, Suite 350, 101 Howard Street, San Francisco, CA, 94105, US

(21) Appl. No.: 10/434,512

(22) Filed: May 8, 2003

Related U.S. Application Data
(60) Provisional application No. 60/378,586, filed on May 8, 2002.

(51) Int. Cl. C12Q 1/68; G01N 33/53; C12M 1/34; G01J 3/00

(52) U.S. Cl. 435/6; 435/7.1; 435/287.2; 356/300

(57) ABSTRACT

The present invention relates to novel components for surface plasmon resonance (SPR) detection of molecular interactions. In particular, the present invention relates to disposable arrayed prisms for use in SPR. The present invention provides improved prisms comprising target biological macromolecules for use in SPR.

This application claims priority to provisional patent application serial No. 60/378,586, filed May 8, 2002, which is herein incorporated by reference in its entirety.

### FIELD OF THE INVENTION

The present invention relates to novel components for label free detection of molecular interactions. In particular, the present invention relates to disposable arrayed prisms for use in surface plasmon resonance (SPR).

### BACKGROUND OF THE INVENTION

Assays for the detection of biological molecules such as nucleic acids or proteins typically involve the use of labeled detection molecules (e.g., fluorescent or radioactive labels). Recent methods utilizing label-free detection have recently been developed.

One such method uses “label-free” detection based on surface plasmon resonance (SPR) for determining the binding of a short oligonucleotide probe to a single-stranded target sequence immobilized to a sensor chip. Since a mismatch significantly affects the binding affinity, the presence of a sequence deviation may be determined. This method has, however, several disadvantages, such as that it requires immobilizing long target sequences, usually PCR products, to the sensor chip and that the sensor chip can not be regenerated.

In using SPR to test for biological, biochemical or chemical substances, a beam of light from a laser source is directed through a prism onto a biosensor consisting of a transparent substrate, usually glass, which has one external surface covered with a thin film of a noble metal, which in turn is covered with an organic film that interacts strongly with an analyte, such as a biological, biochemical or chemical substance. The organic film can contain substances, such as antibodies or antigens, which can bind with an analyte in a sample to cause an increased thickness, which will shift the SPR angle. By either monitoring the position of the SPR angle, or the reflectivity at a fixed angle near the SPR angle, the presence or absence of an analyte in the sample can be detected.

The use of SPR as a testing tool offers several advantages; it is fast, it requires no labeling and it can be done on site. However, to fully achieve these advantages there is a need for a simple, practical biosensor that can be readily modified or adapted to test for a wide variety of analytes, including, biological, biochemical or chemical substances.

### SUMMARY OF THE INVENTION

The present invention relates to novel components for surface plasmon resonance (SPR) detection of molecular interactions. In particular, the present invention relates to disposable arrayed prisms for use in SPR.

Accordingly, in some embodiments, the present invention provides a composition comprising a disposable arrayed prism (e.g., an SPR prism), the prism having an array of biological macromolecules thereon. In some embodiments, the prism further comprises an SPR capable metal film on one face. In some embodiments, the array of biological macromolecules comprises at least 50, preferably at least 100, even more preferably at least 1000, still more preferably at least 10,000, and yet more preferably, at least 100,000 distinct biological macromolecules. In some embodiments, the SPR capable metal film is gold. In some embodiments, the disposable arrayed prism further comprises a plurality of microfluidics channels. In some embodiments, the microfluidics channels are one-dimensional line arrays. In other embodiments, the microfluidics channels are two-dimensional arrays. In some embodiments, the microfluidics channels are fabricated in poly(dimethylsiloxane). In other embodiments, the disposable arrayed prism further comprises a plurality of microchannels etched in the SPR capable metal film. In some embodiments, the biological macromolecule is selected from the group including, but not limited to, nucleic acids, proteins, carbohydrates, and amino acids.

The present invention further provides a system, comprising a composition comprising a disposable arrayed prism (e.g., an SPR prism), the prism having an array of biological macromolecules thereon; and a label free detection (e.g., SPR) apparatus in communication with the disposable arrayed prism. In some embodiments, the prism further comprises an SPR capable metal film on one face. In some embodiments, the array of biological macromolecules comprises at least 50, preferably at least 100, even more preferably at least 1000, still more preferably at least 10,000, and yet more preferably, at least 100,000 distinct biological macromolecules. In some embodiments, the SPR capable metal film is gold. In some embodiments, the disposable array SPR prism further comprises a plurality of microfluidics channels. In some embodiments, the microfluidics channels are one-dimensional line arrays. In other embodiments, the microfluidics channels are two-dimensional arrays. In some embodiments, the microfluidics channels are fabricated in poly(dimethylsiloxane). In other embodiments, the disposable arrayed SPR prism further comprises a plurality of microchannels etched in the SPR capable metal film. In some embodiments, the biological macromolecule is selected from the group including, but not limited to, nucleic acids, proteins, carbohydrates, and amino acids. In some embodiments, the SPR apparatus further comprises a fluid handling device in communication with the microfluidics channels. In some embodiments, the fluid handling device is configured to transfer fluids to the microfluidics channels.

The present invention additionally provides a method of detecting interactions between biological molecules, comprising providing a disposable arrayed prism (e.g., SPR prism), the prism having an array of target biological macromolecules thereon; an apparatus configured for label free (e.g., SPR) detection; and a sample comprising one or more biological molecules; and contacting the sample with the prism and the apparatus under conditions such that the apparatus detects interactions between the target biological molecules and the biological molecules. In some embodiments, the film comprises at least 50, preferably at least 100, even more preferably at least 1000, still more preferably at least 10,000, and yet more preferably, at least 100,000 distinct biological macromolecules. In some embodiments, the SPR capable metal film is gold. In some embodiments, the disposable array SPR prism further comprises a plurality of microfluidics channels. In some embodiments, the microfluidics channels are one-dimensional line arrays. In other embodiments, the microfluidics channels are two-dimensional arrays. In some embodiments, the microfluidics channels are fabricated in poly(dimethylsiloxane). In other embodiments, the disposable arrayed SPR prism further comprises a plurality of microchannels etched in the SPR capable metal film. In some embodiments, the biological macromolecule is selected from the group including, but not limited to, nucleic acids, proteins, carbohydrates, and amino acids. In some embodiments, the nucleic acids are selected from the group including, but not limited to, DNA and RNA. In some embodiments, the SPR apparatus further comprises a fluid handling device. In some embodiments, the fluid handling device is configured to transfer fluids to the microfluidics channels.

### DESCRIPTION OF THE FIGURES

FIG. 1 shows a schematic overview of the arrayed SPR prisms of some embodiments of the present invention.

FIG. 2 shows a final post-hybridization difference image of DNA probes hybridized to arrayed targets on a disposable SPR prism.

FIG. 3 shows two plot profiles taken on a line passing through the center of the arrayed probe spots seen in FIG. 2. The top panel in FIG. 3 is the plot profile before hybridization. The bottom panel is the same profile following hybridization of the complementary oligonucleotide.

FIG. 4 shows the SPR image of arrayed probes hybridized to target oligonucleotides on a disposable prism surface.

FIG. 5 shows the before- and after-hybridization plot profiles of FIG. 3 aligned so that the increase in signal following hybridization is visualized.

FIG. 6 shows the real-time SPR signal resulting from hybridization of the complementary oligonucleotide to the middle spot of the arrayed oligonucleotides.

### DEFINITIONS

As used herein, the term “substrate” refers to any material with a surface that may be coated with a film.

As used herein, the phrase “coated with a film” in regard to a substrate refers to a situation where at least a portion of a substrate surface has a film attached to it (e.g. through covalent or non-covalent attachment).

As used herein, the term “microarray” refers to a solid surface comprising a plurality of addressed biological macromolecules (e.g., nucleic acids or antibodies). The location of each of the macromolecules in the microarray is known, so as to allow for identification of the samples following analysis.

As used herein, the term “disposable arrayed prism” (e.g., “disposable arrayed SPR prism”) refers to a prism that is suitable for use in detection (e.g., SPR detection), comprises an arrayed surface (e.g., a microarray), and is not intended to be reused for multiple detection assays. In some embodiments, the disposable arrayed prisms are those disclosed herein.

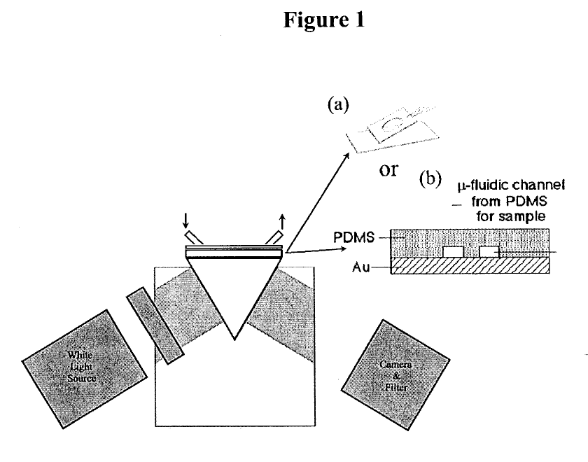

As used herein, the term “coated on one face” when used in reference to an SPR prism, refers to a prism with a coating on one of the main faces of the prism. For example, the triangular prism shown in FIG. 1 is coated on the upward facing surface. The term “face” is not intended to encompass the small facets on each face of a prism that reflect light.

As used herein, the term “SPR capable metal film” refers to any metallic film that is suitable for use in SPR detection. Examples include, but are not limited to, gold, silver, chrome, and aluminum.

As used herein, the term “microfluidics channels” refers to three-dimensional channels created in material deposited on a solid surface. In some embodiments, microchannels are composed of a polymer (e.g., polydimethylsiloxane). Exemplary methods for constructing microchannels include, but are not limited to, those disclosed herein.

As used herein, the term “one-dimensional line array” refers to parallel microfluidic channels on top of a surface that are oriented in only one dimension.

As used herein, the term “two dimensional arrays” refers to microfluidics channels on top of a surface that are oriented in two dimensions. In some embodiments, channels are oriented in two dimensions that are perpendicular to each other.

As used herein, the term “microchannels” refers to channels etched into a surface. Microchannels may be one-dimensional or two-dimensional.

As used herein, the term “biological macromolecule” refers to large molecules (e.g., polymers) typically found in living organisms. Examples include, but are not limited to, proteins, nucleic acids, lipids, and carbohydrates.

As used herein, the term “target molecule” refers to a molecule in a sample to be detected. Examples of target molecules include, but are not limited to, oligonucleotides (e.g. containing a particular SNP), viruses, polypeptides, antibodies, naturally occurring drugs, synthetic drugs, pollutants, allergens, affector molecules, growth factors, chemokines, cytokines, and lymphokines.

The term “sample” as used herein is used in its broadest sense and includes, but is not limited to, environmental, industrial, and biological samples. Environmental samples include material from the environment such as soil and water. Industrial samples include products or waste generated during a manufacturing process. Biological samples may be animal, including, human, fluid (e.g., blood, plasma and serum), solid (e.g., stool), tissue, liquid foods (e.g., milk), and solid foods (e.g., vegetables).

As used herein, the term “test sample” refers to any type of sample (e.g. environmental, industrial, biological, etc.) that is suspected of containing a target molecule.

The term “signal” as used herein refers to any detectable effect, such as would be caused or provided by an assay reaction.

As used herein, the terms “subject” and “patient” refer to any animal, such as a mammal like a dog, cat, bird, livestock, and preferably a human. In preferred embodiments, a subject or patient is the source of a test sample.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5X SSPE (43.8 g/l NaCl, 6.9 g/l NaH₂PO₄ H₂O and 1.85 g/l EDTA, pH adjusted to 7.4 with NaOH), 0.5% SDS, 5X Denhardt's reagent and 100 μg/ml denatured salmon sperm DNA followed by washing in a solution comprising 0.1X SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5X SSPE (43.8 g/l NaCl, 6.9 g/l NaH₂PO₄ H₂O and 1.85 g/l EDTA, pH adjusted to 7.4 with NaOH), 0.5% SDS, 5X Denhardt's reagent and 100 μg/ml denatured salmon sperm DNA followed by washing in a solution comprising 1.0X SSPE, 1.0% SDS at 42° C. when a probe of about 500 nucleotides in length is employed.

“Low stringency conditions” comprise conditions equivalent to binding or hybridization at 42° C. in a solution consisting of 5X SSPE (43.8 g/l NaCl, 6.9 g/l NaH₂PO₄ H₂O and 1.85 g/l EDTA, pH adjusted to 7.4 with NaOH), 0.1% SDS, 5X Denhardt's reagent [50X Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5X SSPE, 0.1% SDS at 42° C. when a probe of about 500 nucleotides in length is employed.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular nucleic acid sequences.

As used herein, the term “target,” refers to a nucleic acid sequence or structure to be detected or characterized. Thus, the “target” is sought to be sorted out from other nucleic acid sequences. A “segment” is defined as a region of nucleic acid within the target sequence.

### DETAILED DESCRIPTION

The present invention relates to novel components for label free detection of molecular interactions. In particular, the present invention relates to disposable arrayed prisms for use in surface plasmon resonance (SPR).

Surface Plasmon Resonance techniques involve a surface coated with a thin film of a conductive metal, such as gold, silver, chrome or aluminum, in which electromagnetic waves, called Surface Plasmons, can be induced by a beam of light incident on the metal glass interface at a specific angle called the Surface Plasmon Resonance angle. Modulation of the refractive index of the interfacial region between the solution and the metal surface following binding of the captured macromolecules causes a change in the SPR angle which can either be measured directly or which causes the amount of light reflected from the underside of the metal surface to change. Such changes can be directly related to the mass and other optical properties of the molecules binding to the SPR device surface. Several biosensor systems based on such principles have been disclosed (See e.g., WO 90/05305).

Generally, in a Kretschman-configuration SPR device, a glass cover slip or slide of appropriate refractive index is coated with a thin (on the order of 50 nm) SPR-capable metal layer. This metal surface is then chemically patterned, and probe molecules are attached to the pattern features. The patterning can be either a basic grid-like array, or microfluidic channels can be overlaid onto the surface for probe deposition and sample application. This gold coated, patterned slide is then optically linked to a prism. This linkage is accomplished by placing a thin film of index-matching fluid between the prism and the slide. A sample solution is then passed over the probes arrayed on the surface. Interaction of an analyte in the solution with a probe molecule on the surface is detected as a change in refractive index. Importantly, SPR detection is label-free.

Using a slide optically coupled to the prism presents several problems, one of which is poor refractive index matching between currently available index matching fluids and high-refractive index glass. Poor index matching results in the appearance of diffraction rings in current images. Also, the matching fluid is difficult to handle, and frequently is plagued by bubbles, leaks, and drying. Furthermore, available matching fluids are often chemically altered by heating. The SPR-ready coated surfaces require careful handling, and assembly of the current prism/matching fluid/slide system uses too many parts and requires too much manipulation for novice users to do readily. With careful handling, approximately 30 cycles of analysis and cleaning can be performed on a single SPR slide. However, uncoupling of the slide from the prism results in contamination of the patterned slide surface with matching fluid and destroys the slide. This means that all 30 cycles of analysis must be carried out in series, with no intervening changes in the arrayed slide.

Accordingly, the present invention provides improved SPR prisms. The prisms of the present invention are relatively cheap in comparison to the large prisms currently being used. The prisms of the present invention overcome the problems of the currently available systems by eliminating the slide as a separate element of the SPR imager's optics. This is accomplished by coating one face of a small low-cost prism with an SPR capable metal film. In some embodiments, the coated surface is then patterned using the same chemistry as used on a metal-coated slide; and can also incorporate the sample-handling and detection advantages of microfluidics. Novice users have little difficulty swapping prisms, since little assembly is required. Such prisms are truly reusable, as removal from the SPR imager and storage does not present any difficulties.

I. SPR Prisms

In some embodiments, the present invention provides improved SPR prisms. The prism may be made of any suitable material including, but not limited to, glass and silica. In preferred embodiments, prisms are made of a high refractive index material. Preferred materials are those whose SPR minimum falls within an angle range. The range can be determined by applying known formulas (See e.g., Hansen, W. N. Journal of the Optical Society of America 53(3):380-390). For example, in some embodiments, prisms are made from a material including, but not limited to, BK-7 glass, SFL-6 glass, and preferably SF-10 glass.

In some embodiments, the SPR prisms of the present invention are disposable. The SPR prisms of the present invention are suitable for single use applications due to lowered material cost (e.g., because of their decreased size). In addition, the prisms aren't integral components of the SPR apparatus, and can be swapped out easily. The prisms of the present invention are, however, suitable for multiple rounds of dybridization/denaturation on each surface).

In other embodiments, prisms are recycled. For example, in some embodiments, used prisms are stripped of all attached materials (including the gold) and reused in new applications (e.g., with different biological macromolecules).

In some embodiments, the prisms are coated on one face with an SPR-capable metal layer. The present invention is not limited to a particular type of metal. Any metal that is suitable for use in SPR may be utilized including, but not limited to, gold, silver, chrome or aluminum. The thickness of the metal film is not overly critical insofar as the film is uniformly applied and will function in SPR imaging analysis. In preferred embodiments, a film of about 450 Å thick is used. In preferred embodiments, gold is utilized as the SPR capable film to coat the prisms.

In some embodiments, the metal (e.g., gold) layer is chemically patterned for attachment of molecular probes (e.g., biomolecules). The present invention is not limited to a particular biological macromolecule. A variety of biological macromolecules are contemplated including, but not limited to, DNA, proteins, carbohydrates, lipids and amino acids.

The present invention is not limited to prisms for SPR. The disposable arrayed prisms of the present invention are suitable for use in a variety of label-free detection systems, including, but not limited to, the label free electrical detection method described in WO 01/61053A2 (herein incorporated by reference) and the oligonucleotide-conjugated nanoparticles described in U.S. Pat. No. 6,361,944, herein incorporated by reference.

II. Arrays

In some embodiments, the metal (e.g., gold) layer is chemically patterned for attachment of molecular probes (e.g., biomolecules). The present invention is not limited to a particular biological macromolecule. A variety of biological macromolecules are contemplated including, but not limited to, DNA, proteins, carbohydrates, lipids and amino acids.

In some embodiments, the present invention further provides prisms comprising arrays of biological macromolecules. In preferred embodiments, arrays comprise at least 50, preferably at least 100, even more preferably at least 1000, still more preferably, at least 10,000, and yet more preferably, at least 100,000 distinct biological macromolecules. In preferred embodiments, each distinct biological macromolecule is addressed to a specific location on the array. In preferred embodiments, each addressable location is larger than 25, and preferably, larger than 50 microns.

The present invention is not limited to a particular method of fabricating or type of array. Any number of suitable chemistries known to one skilled in the art may be utilized.

A. Amine Modified Surface Arrays

In some preferred embodiments, the method of generating arrays described in U.S. Pat. No. 6,127,129 (herein incorporated by reference) is utilized. In the first step of the method, a monolayer of an thiol is self-assembled from an ethanolic solution onto a prism of the present invention, which has been coated with a thin noble-metal film as described above. The present invention is not limited to a particular thiol. A variety of lengths and positions of attachment of the thiol group are contemplated as being suitable for use in the present invention. In some preferred embodiments, long chain (e.g., 11 carbon) alkanethiols are utilized. In other embodiments, branched or cyclic thiols are utilized.

In some embodiments, amine (e.g., MUAM) or carboxylic acid terminated (e.g., MUA), hydroxyl terminated (e.g., MUD), or MUAM modified to be thiol terminated are utilized. In some particularly preferred embodiments, an ω-modified alkanethiol, preferably an amine-terminated alkanethiol, most preferably 11-mercaptoundecylamine (MUAM), is utilized (See e.g., Thomas et al. J Am. Chem. Soc. 117:3830 [1995]).

Self-assembled monolayers of ω-modified alkanethiols on gold form well ordered, monomolecular films. However, if left exposed for extended periods of time, the terminal amine groups of amino-modified alkanthiols may react with CO₂ to form carbamate salts on the surface. Consequently, it is preferred that exposure of amino-terminated alkanethiol-coated substrates to CO₂ be minimized.

Next, the alkanethiol-covered surface is reacted with a reversible protecting group to create a hydrophobic surface. In certain embodiments utilizing an amine-modified alkanethiol such as MUAM, the protecting group is an amino protecting group, preferably 9-fluorenylmethoxycarbonyl (Fmoc). The present invention is not limited to an Fmoc protecting group. Any reversible protecting group may be utilized. Preferred protecting groups offer efficient protection, favorable (e.g., to biological molecules) deprotecting conditions, efficient deprotection, and are suitable for reactions on a surface. For example, in some embodiments, tert-butoxycarbonyl (tBOC) is utilized for the protection of alkanethiols.

Fmoc is a bulky, hydrophobic, base labile, amine protecting group routinely used in the solid phase synthesis of peptides. The choice of protecting group used is dependent in large measure upon the nature of the ω-modification made to the alkanethiol. If the ω-modification is the addition of a carboxyl group, a hydrophobic carboxy protecting group is preferred. Likewise, if the ω-modification is the addition of a hydroxyl or thiol group, a hydrophobic hydroxy or thiol protecting group, respectively, is preferred used. Any type of hydrophobic protecting group suitable for protecting the ω-modification used on the alkanethiol can be utilized in the present invention. Numerous such protecting groups, for any number of reactive moieties, such as amine, hydroxy, ester, carbamate, amides, ethers, thoioethers, thioesters, acetals, ketals and carboxy functionalities, are known to the art (See e.g., Frutos et al., Langmuir 16:2192 [2000]). For example, chloride derivatives of both Fmoc and trityl can be used to reversibly modify hydroxyl-terminated alkanethiols.

In some embodiments utilizing Fmoc protecting groups, the N-hydroxysuccinimide ester of Fmoc (Fmoc-NHS) is reacted with the terminal amine moiety of the MUAM molecule to form a stable carbamate (urethane) linkage, covalently attaching the Fmoc group to the surface.

Subsequently, the bond anchoring the alkanethiol to the metal substrate is selectively cleaved to yield a patterned surface of exposed metal. In some preferred embodiments, UV photopatterning is utilized to create the patterned surface. However, any suitable method of generating a patterned surface may be utilized. For example, in some embodiments, microcontact printing methods can also be used to yield a patterned surface. Using UV patterning, the surface is exposed through a quartz mask to UV radiation, which photo-oxidizes the gold-sulfur bond that anchors the alkanethiol monolayers to the surface. The surface is then rinsed, removing the photo-oxidized alkanethiol and leaving an array of bare metal pads surrounded by a hydrophobic MUAM+Fmoc background. Using photopatterning, features with dimensions as small as 50 mm have been achieved; using microcontact printing methods, arrays with features as small as about 100 nm are achievable.

The surface is next exposed to an alkanethiol solution (in some preferred embodiments, an ethanolic solution of MUAM) whereby the alkanethiol assembles into the bare gold regions producing a surface composed of hydrophilic alkanethiol pads surrounded by the hydrophobic blocked background. This difference in hydrophobicity between the reactive alkanethiol regions and the background is useful for the pinning of small volumes of aqueous biomolecule or cell solutions onto individual array locations.

Biological macromoleucles are then covalently attached to the surface. The alkanethiol active pads are first exposed to a solution of a bifunctional linker. Preferred linkers are those capable of binding at one end to the alkanethiol surface and at the other end to the biological macromolecule to be immobilized to form the desired array. Any bifunctional (e.g., hetero or homo bifunctional) linker having these characteristics can be used in the present invention (See e.g., Smith et al, Langmuir 17:2502 [2001] and the Catalog of Pierce Chemical Company, Rockford, Ill.). Exemplary linkers include, but are not limited to, SSMCC, disuccinimidyl subarate (DSS), and phenyl diisothiocyanate (PDITC).

The preferred bifunctional linker is sulfosuccinimidyl 4-(N-maleimidomethyl)cyclohexane-1-carboxylate (SSMCC), a heterobifunctional linker which contains both an N-hydroxysulfosuccinimide (NHSS) ester and a maleimide functionality. The NHSS ester end of the molecule reacts with the free amine groups on an amino-modified surface, such as the MUAM spots, creating pads terminated in maleimide groups, which are reactive towards thiols. Small volumes (0.08 to 0.1 L) of 1 mM solutions of 5′-thiol-modified biological macromolecules (e.g., DNA sequences) are then spotted at discrete array locations and react to form a covalent attachment to the surface. Using this technique, any number of biological macromolecules can be spotted at different array locations.

The protecting group (e.g., Fmoc) is next removed from the array surface. Preferably, this is accomplished by exposure to a 1M solution of the secondary amine, TAEA, in DMF. Many basic secondary amines can be used to remove Fmoc from the surface (e.g., including, but not limited to, 1M solutions of ethanolamine and piperidine). After the deprotection step, the array background has been converted back to the original alkanethiol surface.

In the final step of the array fabrication, the alkanethiol background is reacted with a compound to create a background that is resistant to the non-specific binding of proteins. The preferred compound for this purpose is PEG-NHS, although any compound that will selectively bind to the alkanethiol surface and inhibit non-selective protein binding can be used. In order to effectively monitor the binding of proteins to arrays of surface-bound biomolecules or cells, it is preferred that the array background prohibit the non-specific adsorption of protein molecules. Additional blocking groups include, but are not limited to, mixtures of PEG-terminated and other molecules (e.g., hydroxyl-terminated), different molecular weights of PEG molecules, polylysine, casein, BSA, and octadecane thiol (See e.g., Chapman et al., J. Am. Chem. Soc., 122:8303 [2000]).

B. Additional Arrays

The present invention is not limited to the array fabrication methods described above. Additional array generating technologies may be utilized, including, but not limited to, those described below.

In some embodiments, a DNA array is generated using photolithography on a prism surface (Affymetrix, Santa Clara, Calif.; See e.g., U.S. Pat. Nos. 6,045,996; 5,925,525; and 5,858,659; each of which is herein incorporated by reference) assay. The technology uses miniaturized, high-density arrays of oligonucleotide probes affixed to the prism. Probe arrays are manufactured by Affymetrix's light-directed chemical synthesis process, which combines solid-phase chemical synthesis with photolithographic fabrication techniques employed in the semiconductor industry. Using a series of photolithographic masks to define exposure sites, followed by specific chemical synthesis steps, the process constructs high-density arrays of oligonucleotides, with each probe in a predefined position in the array.

In other embodiments, a DNA array containing electronically captured probes (labeled nucleic acid sequences) (Nanogen, San Diego, Calif.) is utilized (See e.g., U.S. Pat. Nos. 6,017,696; 6,068,818; and 6,051,380; each of which are herein incorporated by reference). In some embodiments, a modified method of Nanogen's technology, which enables the active movement and concentration of charged molecules to and from designated test sites on a semiconductor microchip is utilized. DNA capture probes are electronically placed at, or “addressed” to, specific sites on the prism. Since DNA has a strong negative charge, it can be electronically moved to an area of positive charge.

First, a test site or a row of test sites on the prism is electronically activated with a positive charge. Next, a solution containing the DNA probes is introduced onto the prism. The negatively charged probes rapidly move to the positively charged sites, where they concentrate and are chemically bound to a site on the prism. The prism is then washed and another solution of distinct DNA probes is added until the array of specifically bound DNA probes is complete.

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the prism by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and removing by spinning.

DNA probes unique for the target sequence of interest are affixed to the prism using Protogene's technology. The prism is then contacted with a test sample of interest. Following hybridization, unbound DNA is removed and hybridization is detected using SPR.

III. Microfluidics

In some embodiments, arrays are fabricated by patterning the prism with microfluidic channels. In some embodiments, microfluidics are generated using the polydimethylsiloxane (PDMS) polymer-based methods described by Lee et al. (Analytical Chemistry, 73:5525 [2001]). This technique can be used for both fabricating 1-D DNA microarrays using parallel microfluidic channels on chemically modified gold and silicon surfaces, and in a microliter detection volume methodology that uses 2-D DNA microarrays formed by employing the 1-D DNA microarrays in conjunction with a second set of parallel microfluidic channels for solution delivery.

For example, in some embodiments, microliter detection volume methodology that uses 2-D DNA hybridization microarrays formed by employing 1-D DNA line arrays in conjunction with a second set of parallel microfluidic channels for solution delivery is utilized. In some embodiments, PDMS microchannels are fabricated by replication from 3-D silicon wafer masters that were created photolithographically from 2-D chrome mask patterns (See e.g., Duffy et al., Anal. Chem., 70:4974 [1998] and Effenhauser et al., Anal. Chem., 69:3451 [1997]).

A gold thin film surface deposited on the SPR prism is reacted with MUAM in order to form a self-assembled monolayer on the gold surface as described above. A PDMS polymer film containing parallel microchannels is then attached to the MUAM modified gold surface. In some embodiments, a surface pattern is created by flowing the heterobifunctional linker SSMCC through the PDMS microchannels over the gold surface. The SSMCC reacts with the MUAM to create a maleimide-terminated alkanethiol monolayer. Biological macromolecules (e.g., 5′-thiol-modified DNA or RNA probes) are than each flowed into a separate PDMS microchannel and react with the maleimide-terminated gold surface to form an array of probes on the surface of the gold. In some embodiments, the microchannels are cleaned with water, the PDMS is removed from the surface and the gold slide is soaked in a PEG-NHS solution in order to modify the MUAM background (see above description of blocking with PEG-NHS). The PEG-coated background helps to eliminate nonspecific adsorption of DNA or RNA during hybridization experiments.

The present invention is not limited to a particular method of fabricating channels in the prisms of the present invention. For example, in other embodiments, the present invention utilizes microchannels etched into the prism (See e.g., U.S. Pat. No. 6,176,962, herein incorporated by reference). In still further embodiments, microfluidic channels are fabricated using wet chemical etching (Wang et al., Anal. Chem., 72:2514 [2000]) or soft lithography (Deng et al., Anal. Chem. 72:3176 [2000]).

IV. Assembly of Prisms

In some embodiments, following patterning or generation of arrays, a silicone gasket (Grace Biolabs, Bend, Oreg.) is sandwiched in-between a gold-coated prism and a microscope cover slide to form a small reaction chamber used with SPR (shown in FIG. 1(a)). In other embodiments, a HYBRIWELL seal (Grace Biolabs) is used to create a low-volume reaction chamber.

V. Kits and Systems

In certain embodiments, the present invention provides kits and systems for making and using the SPR prisms of the present invention. In some embodiments, the kits and systems comprise various components for generating the SPR prisms of the present invention (e.g. gold solutions, solutions for generating one or two dimensional arrays, solutions for adding reactive groups to the surface of the prisms, biological molecules, buffers and other components used to make a prism of the present invention). In this regard, any assortment of components may be assembled into a system or kit, such that, for example, a user may use such kits to generate coated SPR prisms of the present invention. These systems and kits may also include instructions for employing the components of the system or kit to generate the prisms of the present invention. The kits and systems of the present invention may also comprise SPR prisms already coated with biological macromolecules along with various other reaction components that may be employed to perform detection reactions with the prisms of the present invention. For example, kits of the present invention may comprises buffers, a reaction chamber to seal the microarray from the outside environment (e.g., the silicon gaskets described above), control target samples (e.g., known to contain the target), etc. Instructions for employing the prisms of the present invention may also be included.

In other embodiments, the present invention provides a system comprising an SPR prism of the present invention and an apparatus for performing SPR. In some embodiments, the apparatus comprises a solid-state light source for an SPR imaging setup. A light source is needed for the creation of surface plasmons in the SPR experiment. In 1999, Nelson et al. (Analytical Chem., 71:3928 [1999]) described the use of a collimated white light source for SPR imaging. This SPR imaging system (now manufactured by GWC Instruments) uses an incandescent bulb as a light source. The light is captured and collimated by a series of lenses and pinholes to homogeneously illuminate a 2-cm diameter circular area in which the sample is placed. The ideal light source for creation of a collimated, homogeneous beam of light is a single point source. However, an incandescent bulb produces light through its glowing wound filament wire, far from a single point source. This makes creation of homogeneous illumination difficult using an incandescent bulb. This can be alleviated to some extent through careful and time-consuming alignment of the incandescent bulb. However, incandescent light bulbs have a limited lifetime and must be replaced. Replacement bulbs must be realigned every time they are replaced. In some embodiments, the present invention provides an alternative is to use solid-state illumination, such as an LED, LED array, fiber optic, or light pipe. This can be used to produce either a high quality single-point source, or a diffuse source for the SPR imager. These light sources last much longer than standard bulbs, and are very flexible.

VI. Detection Assays

The arrayed prisms of the present invention are preferably employed for detecting the presence or absence of target molecules (e.g. target molecules in a test sample). For example, in some embodiments, one component of a detection assay (e.g. antibody or oligonucleotide) is attached to a prism. In certain embodiments, the test sample is contacted with the arrayed prisms of the present invention and various operations are carried out, such as the addition of miscellaneous reagents, incubations, washings, and the like. In this regard, arrayed SPR prisms of the present invention may carry out thousands of detection reactions (e.g., DNA detection assays, etc.) to determine if target molecules are present in a test sample. In preferred embodiments, detection is label free SPR detection.

In some preferred embodiments, 2-dimensional microfluidic arrayed prisms are utilized in detection assays. In certain embodiments, the second channel of the 2-D microarray is used to deliver sample (e.g., sample suspected of a containing a particular nucleic acid or other biological molecule), as well as buffers or other hybridization solutions. The interaction of biological molecules with molecules on the microarray is then detected using SPR.

In some embodiments, a standard SPR detection apparatus is used in the detection of bound nucleic acids (See e.g., FIG. 1 and the above description). In other embodiments, the apparatus includes an LED light source (see above). In further embodiments, the apparatus comprises a fluid handling device (e.g., a pump) for use is delivering solutions to the microfluidic channels.

EXPERIMENTAL

The following examples are provided in order to demonstrate and further illustrate certain preferred embodiments and aspects of the present invention and are not to be construed as limiting the scope thereof.

### EXAMPLE

### SPR Imaging of DNA Targets Hybridized to Arrayed Probes on a Prism Surface

A MUAM surface was created on an SPR-capable gold coated glass prism as described above. Briefly, 45 nm thick SPR-capable gold thin film was vapor-deposited over a 0.5 nm chromium adhesion layer that had been vapor-deposited on an SF-10 glass prism. A MUAM monolayer was deposited on the thin gold film from an ethanolic MUAM solution. The heterobifunctional crosslinker SSMCC was reacted with the amine surface, creating a thiol-reactive maleimide surface. The surface was rinsed to remove unreacted SSMCC with distilled water. Approximately 0.4 ul of thiol modified oligonucleotide probe (5′ thiol TTT TTT TTT TTT TTT GAT CGA ACT GAC CGC CCG CGG CCC GT 3′; SEQ ID NO:1), 1 mM in 20 mM phosphate buffered pH 7.4, were spotted onto the SSMCC surface by hand using a micropipettor. The surface was placed in a humid chamber and incubated 12 hours to affect reaction of the thiol DNA probe to the maleimide (SSMCC) surface.

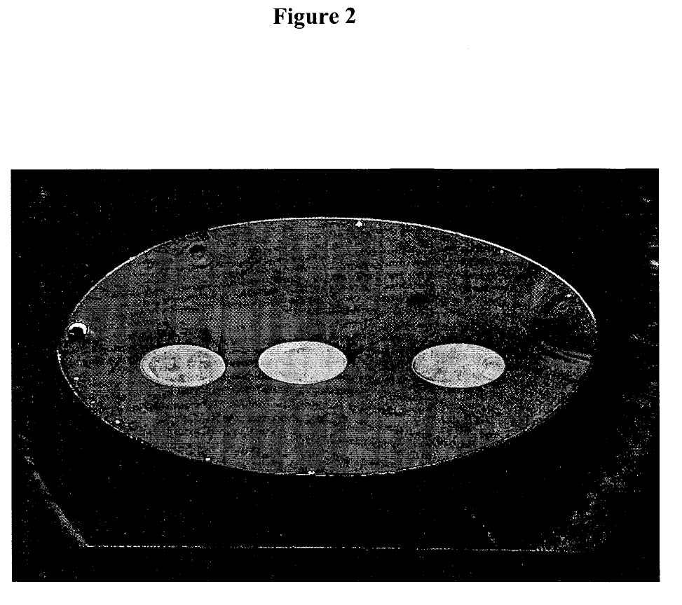

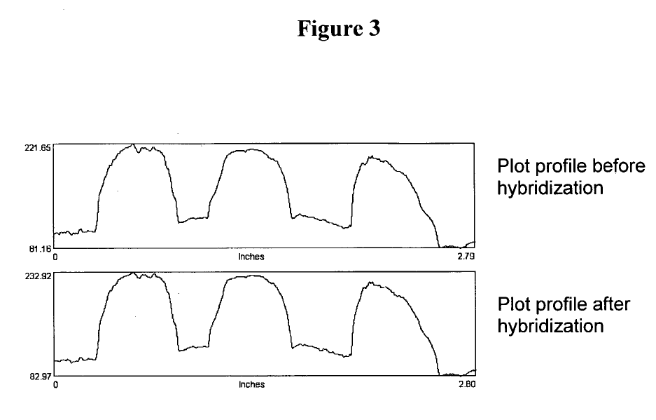

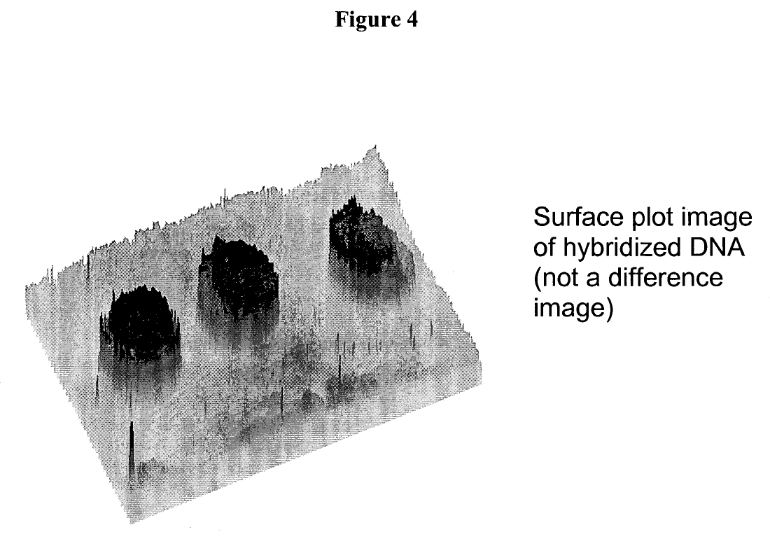

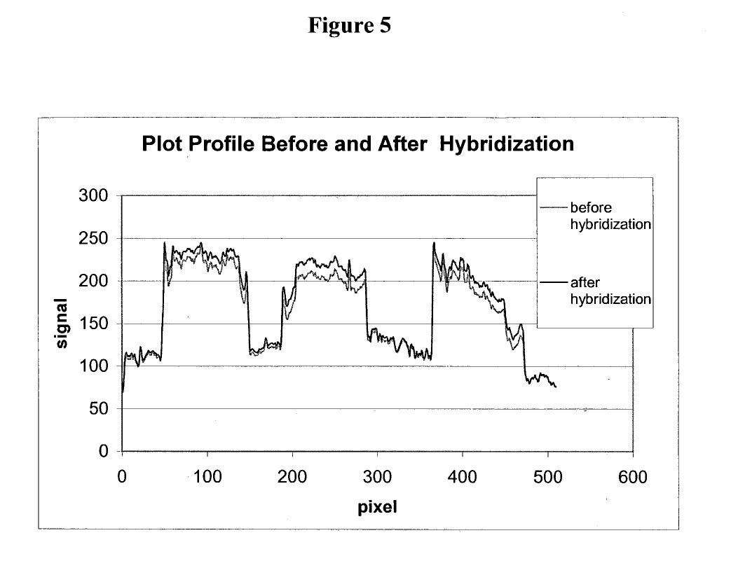

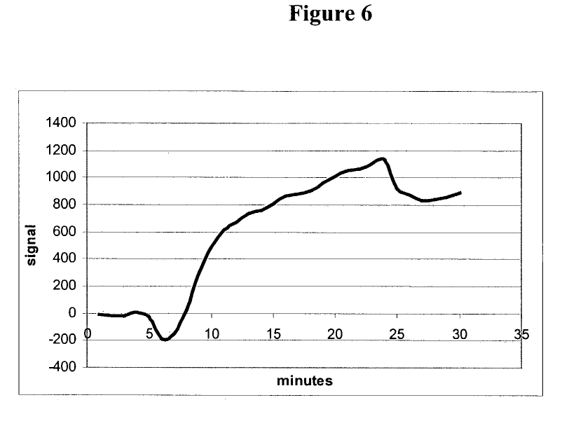

The prism with arrayed DNA probes attached to the surface was placed in the SPR imager, and hybridization buffer was flowed over the surface for several minutes to equilibrate the surface. The complementary DNA oligonucleotide (5′ ACG GGC CGC GGG CGG TCA GTT CGA TC 3′; SEQ ID NO:2) was flowed in at a concentration of 2 micromolar in hybridization buffer. Hybridization was detected with the SPR imager on the regions modified with the thiol probe DNA, and minimal increase in background signal was observed in the background regions lacking probe DNA. FIG. 2 shows the final post-hybridization difference image. The arrayed DNA probe spots with hybridized complementary oligonucleotide were detectable. FIG. 3 shows two plot profiles taken on a line passing through the center of the arrayed probe spots seen in FIG. 2. The top panel in FIG. 3 is the plot profile before hybridization. The arrayed probes were detectable on the prism surface, as evidenced by the three peaks in the profile. The bottom panel is the same profile following hybridization of the complementary oligonucleotide. Hybridization led to a detectable increase in SPR signal. The SPR image of arrayed probes hybridized to target oligonucleotides on a disposable prism surface is shown in FIG. 4. This is the final post-hybridization image, and the signal intensity of the peaks is due to the contribution of both the arrayed probes and the hybridized oligonucleotide. FIG. 5 shows the before- and after-hybridization plot profiles of FIG. 3 aligned so that the increase in signal following hybridization is easily visualized. FIG. 6 shows the real-time SPR signal resulting from hybridization of the complementary oligonucleotide to the middle spot of the arrayed oligonucleotides. An area outside of the array spots was used to standardize the background. Signal from non-specific binding of oligonucleotide complement to the background was subtracted from the SPR signal of the arrayed spot. Thus, the real-time signal change shown in FIG. 6 was due to hybridization of the complement to the arrayed probe, and not due to nonspecific adsorbtion of the complement oligonucleotide to the coated disposable prism surface. Complement was flowed into the reaction cell at 7 minutes. A slight difference in refractive index of the complement solutions caused the drop in signal apparent at the 7 minute mark. Following this initial signal drop, signal increase during the following 25 minute room-temperature hybridization reaction was apparent. The drop in signal after 25 minutes was due to a final rinse with fresh hybridization buffer without complementary oligonucleotide.

These results demonstrate the ability to create an SPR-capable metal coated disposable prism, array biomolecular probes on that prism, and image interactions occurring between the arrayed probes and target molecules.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, or related fields are intended to be within the scope of the following claims:

We claim:

1. A composition comprising a disposable arrayed prism configured for use in label free detection, said prism having an array of biological macromolecules thereon.

2. The composition of claim 1, wherein said label free detection comprises SPR.

3. The composition of claim 2, wherein said prism has an SPR capable metal film on one face.

4. The composition of claim 1, wherein said array of biological macromolecules comprises at least 50 distinct biological macromolecules.

5. The composition of claim 1, wherein said array of biological macromolecules comprises at least 100 distinct biological macromolecules.

6. The composition of claim 1, wherein said array of biological macromolecules comprises at least 1000 distinct biological macromolecules.

7. The composition of claim 3, wherein said SPR capable metal film is gold.

8. The composition of claim 1, wherein said disposable arrayed prism further comprises a plurality of microfluidics channels.

9. The composition of claim 8, wherein said microfluidics channels are one-dimensional line arrays.

10. The composition of claim 8, wherein said microfluidics channels are two-dimensional arrays.

11. The composition of claim 8, wherein said microfluidics channels are fabricated in poly(dimethylsiloxane).

12. The composition of claim 3, wherein said disposable arrayed prism further comprises a plurality of microchannels etched in said SPR capable metal film.

13. The composition of claim 1, wherein said biological macromolecule is selected from the group consisting of nucleic acids, proteins, carbohydrates, and amino acids.

14. A system, comprising a composition comprising a disposable arrayed prism, said prism having an array of biological macromolecules thereon; and a detection apparatus in communication with said disposable arrayed prism, and wherein said prism is configured for label free detection.

15. The system of claim 14, wherein said label free detection comprises SPR.

16. The system of claim 14, wherein said prism has an SPR capable metal film on one face.

17. The system of claim 14, wherein said array of biological macromolecules comprises at least 50 distinct biological macromolecules.

18. The system of claim 14, wherein said array of biological macromolecules comprises at least 100 distinct biological macromolecules.

19. The system of claim 14, wherein said array of biological macromolecules comprises at least 1000 distinct biological macromolecules.

20. The system of claim 16, wherein said SPR capable metal film is gold.

21. The system of claim 14, wherein said disposable arrayed prism further comprises a plurality of microfluidics channels.

22. The system of claim 21, wherein said microfluidics channels are one-dimensional line arrays.

23. The system of claim 21, wherein said microfluidics channels are two-dimensional arrays.

24. The system of claim 21, wherein said microfluidics channels are fabricated in poly(dimethylsiloxane).

25. The system of claim 16, wherein said disposable arrayed prism further comprises a plurality of microchannels etched in said SPR capable metal film.

26. The system of claim 14, wherein said biological macromolecule is selected from the group consisting of nucleic acids, proteins, carbohydrates, and amino acids.

27. The system of claim 14, wherein said detection apparatus is an SPR detection apparatus.

28. The system of claim 27, wherein said SPR apparatus further comprises a fluid handling device in communication with said microfluidics channels.

29. The system of claim 28, wherein said fluid handling device is configured to transfer fluids to said microfluidics channels.

30. A method of detecting interactions between biological molecules, comprising:
a) providing
i) a disposable arrayed prism having an array of target biological macromolecules thereon;
ii) an apparatus configured for label-free detection; and
iii) a sample comprising one or more biological molecules; and
b) contacting said sample with said prism and said apparatus under conditions such that said apparatus detects interactions between said target biological molecules and said biological molecules.

31. The method of claim 30, wherein said disposable array prism is a disposable arrayed SPR prism, and wherein said prism has an SPR capable metal film on one face.

32. The method of claim 30, wherein said prism comprises at least 50 distinct target biological macromolecules.

33. The method of claim 30, wherein said prism comprises at least 100 distinct target biological macromolecules.

34. The method of claim 30, wherein said prism comprises at least 1000 distinct target biological macromolecules.

35. The method of claim 31, wherein said SPR capable metal film is gold.

36. The method of claim 31, wherein said disposable arrayed SPR prism further comprises a plurality of microfluidics channels.

37. The method of claim 36, wherein said microfluidics channels are one-dimensional line arrays.

38. The method of claim 36, wherein said microfluidics channels are two-dimensional arrays.

39. The method of claim 36, wherein said microfluidics channels are fabricated in poly(dimethylsiloxane).

40. The method of claim 31, wherein said disposable arrayed SPR prism further comprises a plurality of microchannels etched in said SPR capable metal film.

41. The method of claim 30, wherein said biological macromolecule is selected from the group consisting of nucleic acids, proteins, carbohydrates, and amino acids.

42. The method of claim 41, wherein said nucleic acids are selected from the group consisting of DNA and RNA.

43. The method of claim 30, wherein said label free detection apparatus is an SPR apparatus.

44. The method of claim 43, wherein said SPR apparatus further comprises a fluid handling device.

45. The method of claim 44, wherein said fluid handling device is configured to transfer fluids to said microfluidics channels.


SEQUENCE LISTING (2 sequences)

SEQ ID NO: 1 - DNA, 41 bp, artificial
  synthetic
tttttttttttttttgatcgaactgaccgcccgcggcccgt

SEQ ID NO: 2 - DNA, 26 bp, artificial
  synthetic
acgggccgcgggcggtcagttcgatc